(19) United States
(12) Patent Application Publication
(10) Pub. No.: US 2019/0167469 A1
(43) Pub. Date: Jun. 6, 2019

(54) COLD TREATMENT APPARATUS

(71) Applicant: FREEZE N FIT INC., Glen Cove, NY (US)

(72) Inventor: Avi SIVAN, Glen Cove, NY (US)

(73) Assignee: FREEZE N FIT INC., Glen Cove, NY (US)

(21) Appl. No.: 16/207,996

(22) Filed: Dec. 3, 2018

Related U.S. Application Data
(60) Provisional application No. 62/593,657, filed on Dec. 1, 2017.

(51) Int. Cl. A61F 7/00

(52) U.S. Cl. CPC A61F 7/007; A61F 2007/0064; A61F 2007/0078; A61F 2007/0086; A61F 2007/0087

(57) ABSTRACT

There is a chiller for cooling a human body down to dissolve fat on a person's body. There is a device which includes a housing. The housing has at least one handle, and is configured to at least partially house a cold plate. The cold plate is also chilled by a chiller disposed inside of the housing. The chiller is at least partially disposed in the housing and is configured to have access to at least one vent. The at least one vent is positioned on the housing. In at least one embodiment, the at least one vent is positioned opposite the cold plate.

### CROSS REFERENCE TO RELATED APPLICATIONS

This application is a non-provisional application that claims priority from provisional application Ser. No. 62/593,657 filed on Dec. 1, 2017, the disclosure of which is hereby incorporated herein by reference in its entirety.

### BACKGROUND OF THE INVENTION

At least one embodiment of the invention relates to a chiller for cooling a human body down to dissolve fat on a person's body.

### SUMMARY OF THE INVENTION

At least one embodiment of the invention relates to a chiller for cooling a human body down to dissolve fat on a person's body. There is a device which includes a housing. The housing has at least one handle, and is configured to at least partially house a cold plate. The cold plate is also chilled by a chiller disposed inside of the housing. The chiller is at least partially disposed in the housing and is configured to have access to at least one vent. The at least one vent is positioned on the housing. In at least one embodiment, the at least one vent is positioned opposite the cold plate.

The housing can have two handles and the vent can have at least two wings as well.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 is a front perspective view of the cold treatment device;

FIG. 2 is a back-perspective view of the cold treatment device;

FIG. 3 is a back-top perspective view of the cold treatment device;

FIG. 3 is a top view of the cold treatment device;

FIG. 4 is a front view of the device;

FIG. 5 is a back view of the device; and

FIG. 6 is a right-side view of the device;

FIG. 7 is a left-side view of the device;

FIG. 8 is a top view of the device;

FIG. 9 is a bottom view of the device;

FIG. 10 is a top view of another embodiment of the device;

FIG. 11 is a back view of the device with the components in dash-dotted lines; and

FIG. 12A is a first open view of the device with the chiller plate and chiller removed showing a fan; and

FIG. 12B is another open view of the device with the chiller being shown and the chiller plate being removed.

### DETAILED DESCRIPTION OF PREFERRED EMBODIMENTS

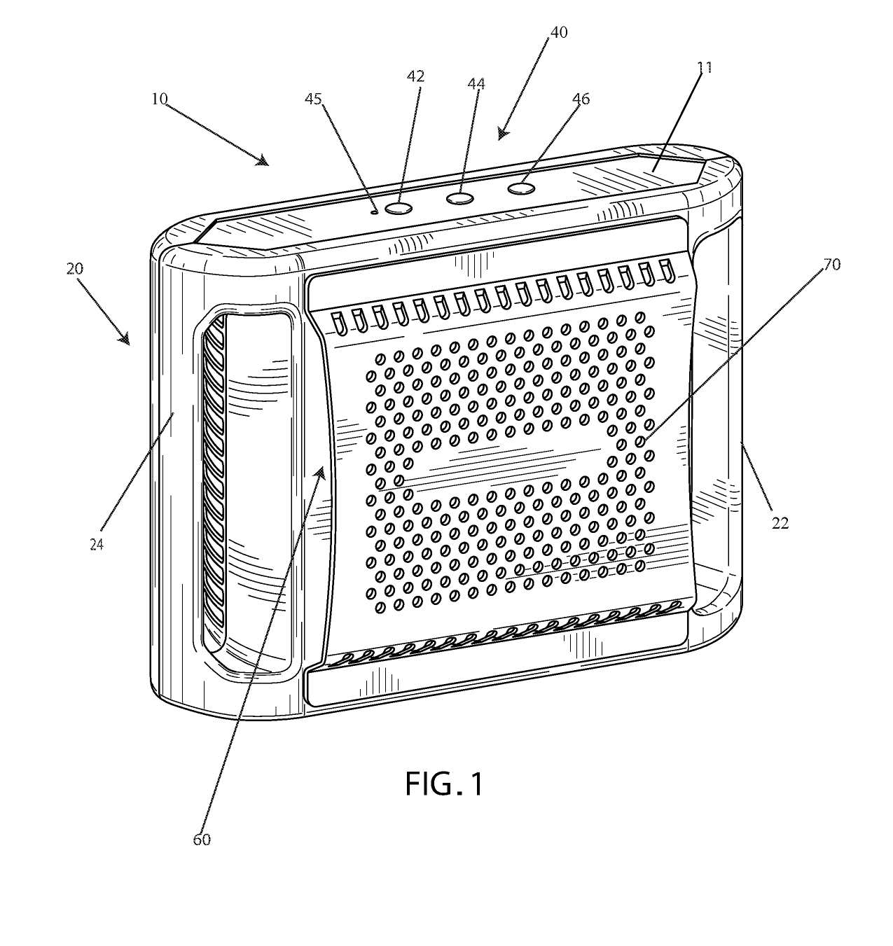

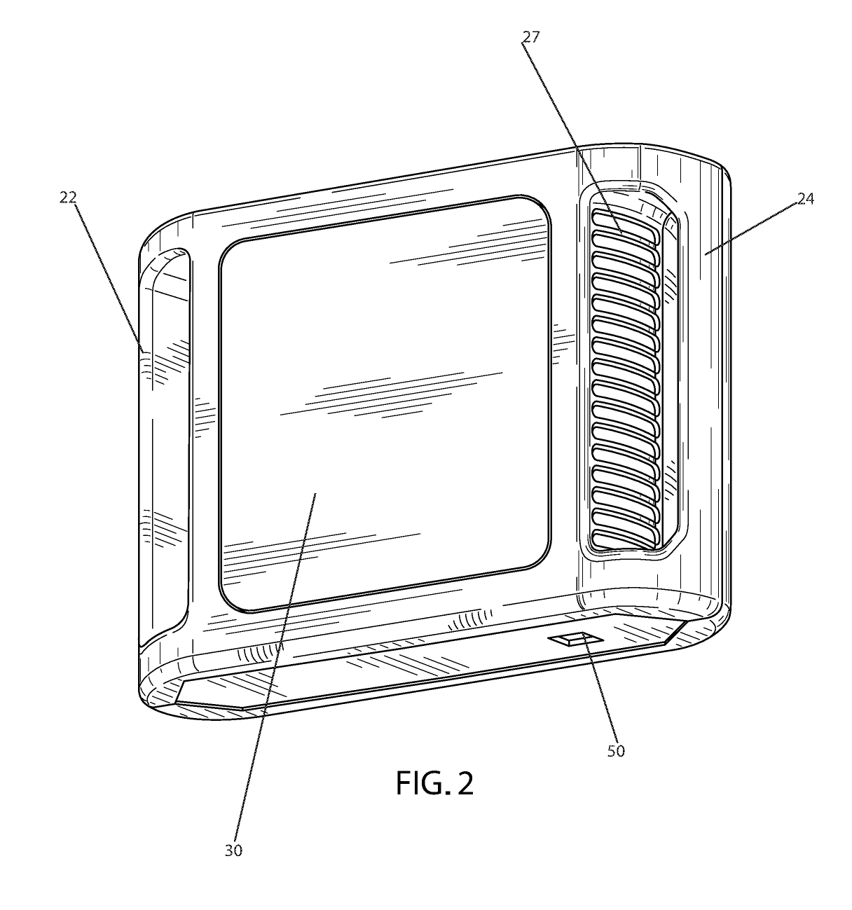

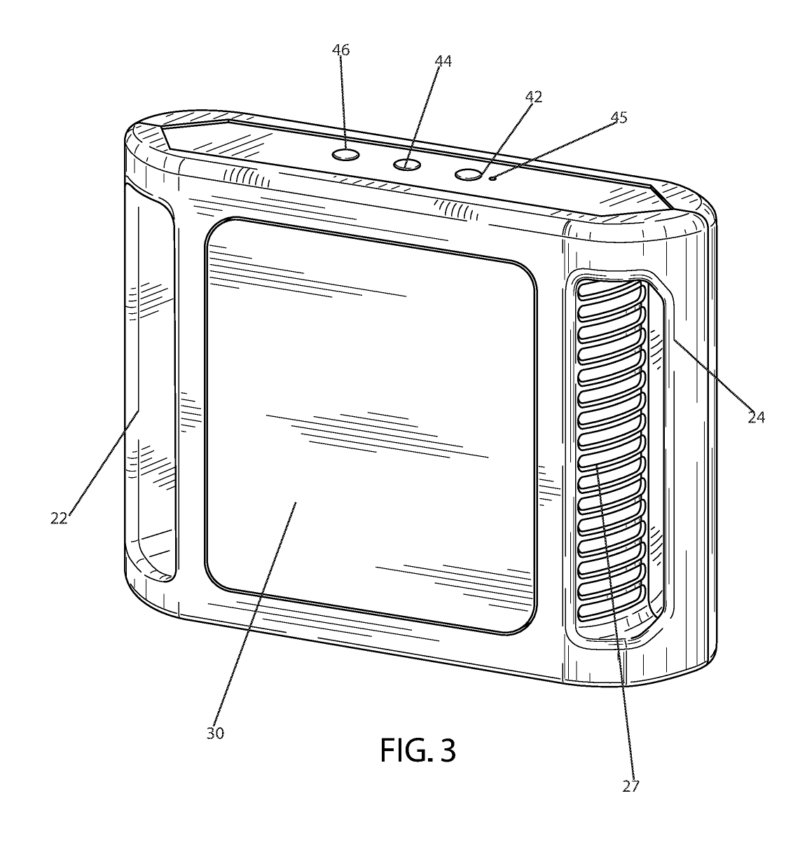

FIG. 1 is a front view of the cold treatment device. For example, in this embodiment, there is shown a first embodiment 10 having a body or housing 11. Body or housing 11 can be made from any suitable material such as a plastic or polymer material. Other materials may be used such as a composite, rubber, or metal as well. The housing is configured to at least partially house at least one cold plate 30 (See FIG. 2). The housing is also configured to have at least two different handles 20 comprising a first handle 22 and a second handle 24. There are gaps 26 and 28 (See FIG. 5) which allow a user to place his hands around the handles 22 and 24. These handles can also be used to receive at least one strap as well. The strap 100 (See FIG. 10) is shown in greater detail in FIG. 3. There is also a controller 40 which has buttons to raise and lower the temperature and a plug connection or power port 50 (see FIG. 2) for receiving power from a power supply. For example, there is an on/off button 42, an up button 44, and a down button 46. On/off button 42 is also associated with light 45 to indicate whether the device is turned on or off.

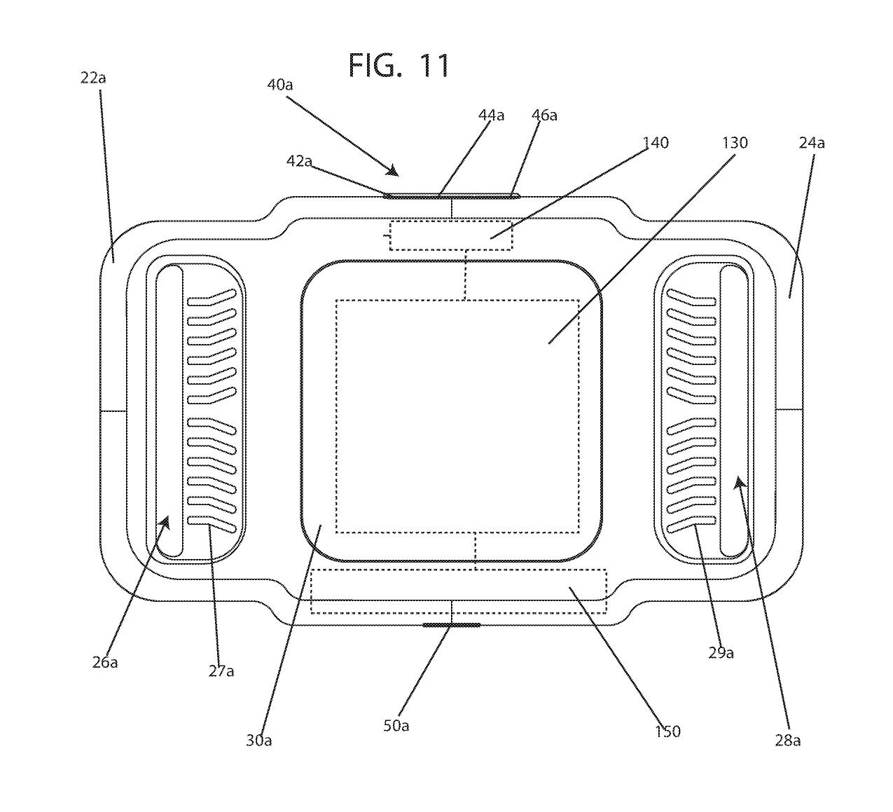

FIG. 2 is a back view of the cold treatment device. In this view there is shown back side which has vents 27. These vents 27 and 29 (see FIG. 5) disposed near the gap regions 26 and 28. These vents are disposed on the first side adjacent to the cold plate 30. These vents are configured to displace heated air from the chiller 130 (See FIG. 11). The vents as described above are configured to vent air or other cooling fluid from the heat exchanger/chiller 130. In addition, in this view there is shown power supply port/plug connection 50 and buttons for controller 40.

FIG. 3 shows a top view of the device which shows controller 40 having buttons including an on/off button 42, an up button 44 and a down button 46. The up button 44 is configured to make the chill plate 30 cooler, while the down button is configured to make the chill plate warmer. In addition, on the back side of the device, there is shown vents 70 as well as wings 62 and 64 which are configured to be spaced from handles 22 and 24.

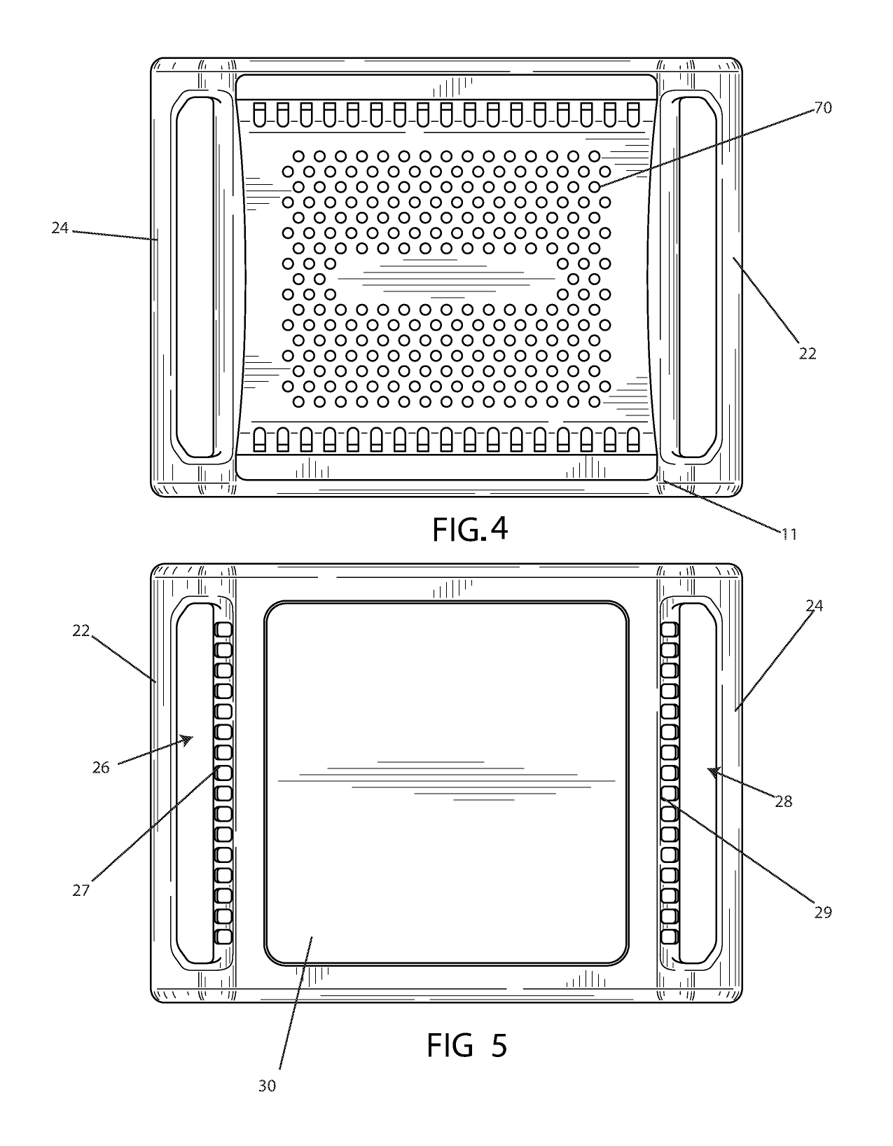

FIG. 4 is a front view of the chiller device which shows handles 22 and 24 as well as a front grill 70. The front grill 70 can be extended out in front of the body of the device leaving a space or a gap between the grill and the body 11.

FIG. 5 is a view of the chiller side of the device with chiller plate 30 being shown. This view shows vents 27 and 29 as well as gaps 26 and 28 between respective handles 22 and 24. The chiller plate can be made from any suitable material such as a metal such as copper, aluminum, or any other type of suitable material.

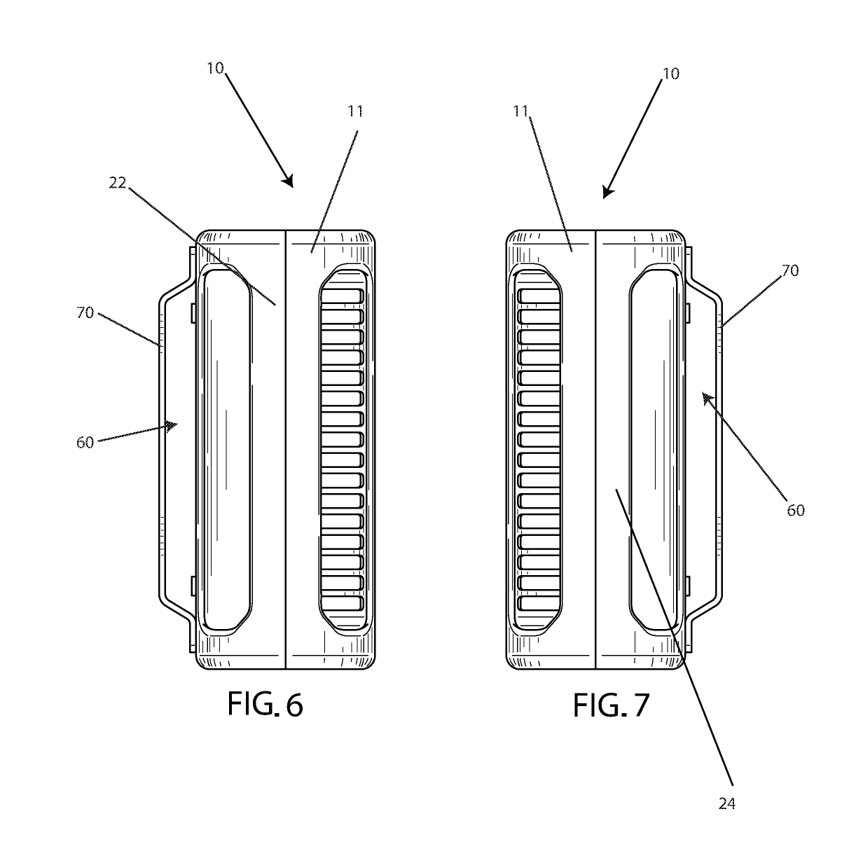

FIG. 6 is a right side view of the device 10 showing handle 22 and grill 70. In addition, there is shown a gap 60 between grill 70 and body 11. FIG. 7 shows a left side view as well which shows handle 24 instead of handle 22 as well as grill 70 body 11, gap 60.

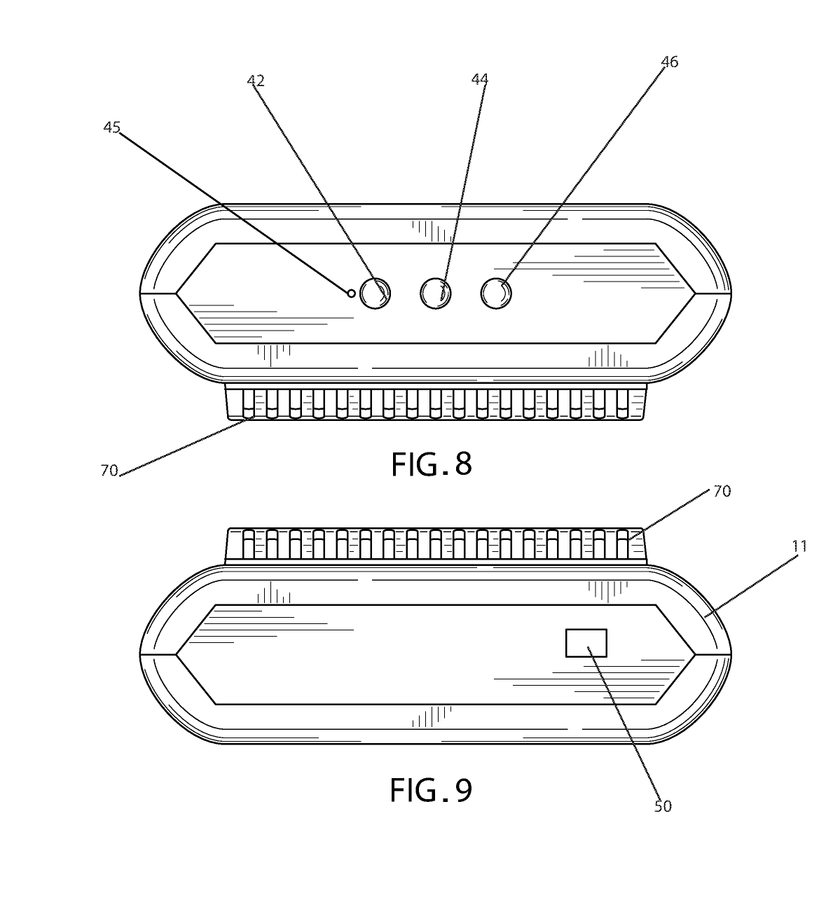

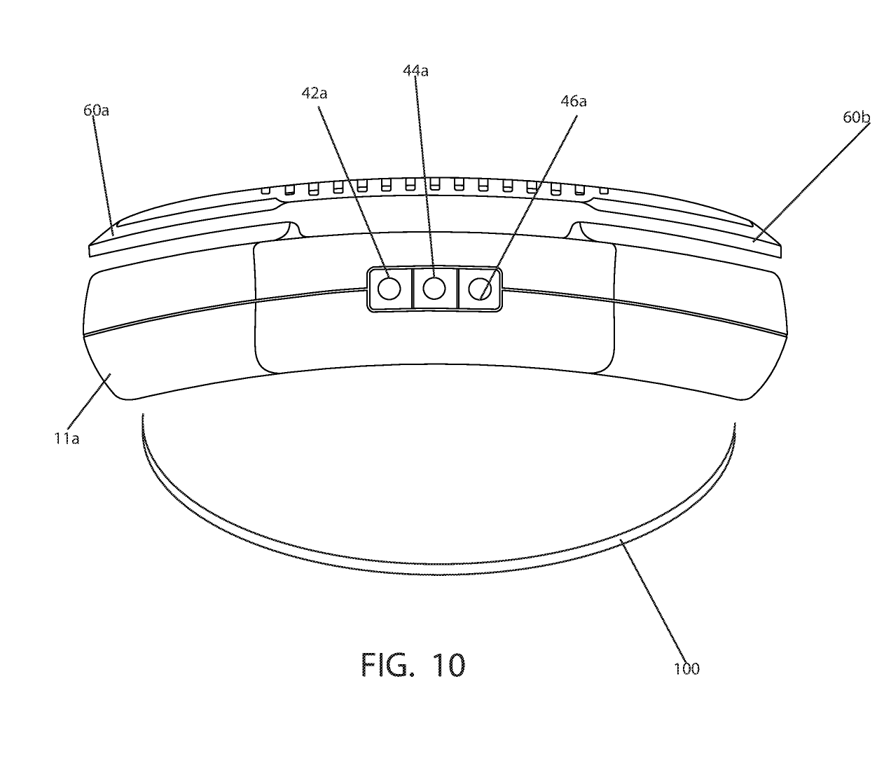

FIG. 8 is a top view of the device 10 including body 11, grill 70, an on/off switch or button 42, and up switch or button 44, and a down switch or button 46. The on/off button is in communication with light 45, wherein this light is used to indicate whether the device is on or off. FIG. 9 is a bottom view which shows a power port 50 which allows the device to be connected to a power supply so that the device can be charged/recharged.

FIG. 10 is a top view of another embodiment, 10a, which shows another body design of a housing 11a, as well as a grill having wings 60a and 60b that are spaced apart from the body or housing 11a. There is an on/off button 42a, an up button 44a, and a down button 46a as well. There is also a strap 100 which can be coupled to the device, wherein this strap can be coupled around the handles (not shown but similar to handles 22 and 24 in FIG. 1).

FIG. 11 is a front view of the device which shows some of the inner electronic components. For example, there is shown chiller 130 which is positioned adjacent to cold plate 30a. Cold plate 30a is configured to be placed on a person's body such as on their stomach or thigh area to essentially chill away the fat on a person's body. A controller 140 is positioned adjacent to controller/buttons 40a such that when the controller/buttons 40a are pressed, it either activates chiller 130 or cools chiller to a lower temperature or warms chiller 130 up to a higher temperature depending on which button is pressed. In addition, disposed inside this housing 11 a is a battery 150 which is configured to store energy from the battery plug connection 50a. These internal components such as chiller 130, controller 140 and battery 150 are present in both the embodiment 10a as well as in the embodiment 10. Vents 27a and 29a are shown as curved vents which are angled instead of straight.

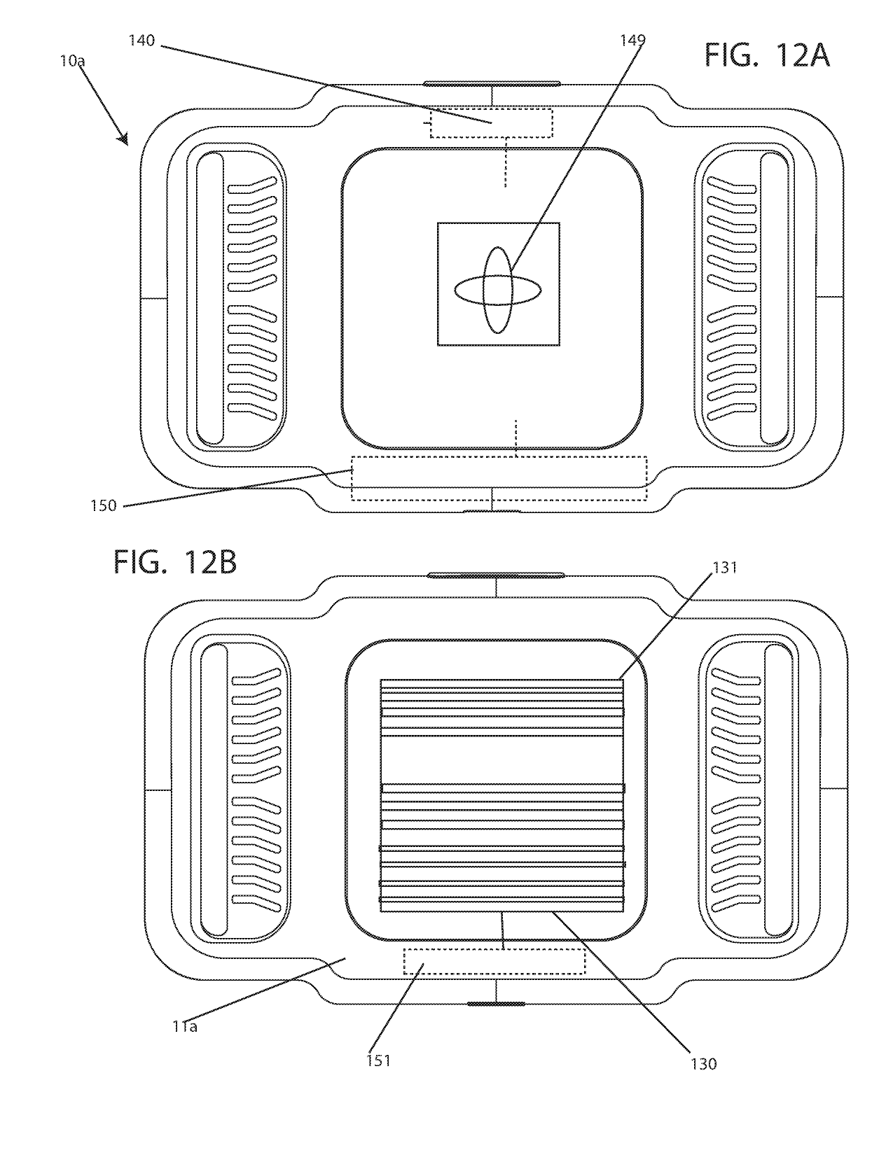

As shown in FIG. 12A, the device can also have a fan 149 disposed behind the chiller 130. Chiller 130 can be in the form of an aluminum or other type of conductive metal which is formed as a ribbed surface shown in FIG. 12B. Each of the ribs 131 of chiller is offset by an empty channel inside. Both the chiller and the fan can be powered by either a battery 150 or a transformer 151 (See FIG. 12B) coupled to an AC power source for direct electrical power.

This device can be constructed so as to be easy to use, portable and adaptable so as to accommodate straps and other devices as well.

Accordingly, while at least one embodiment of the present invention have been shown and described, it is to be understood that many changes and modifications may be made thereunto without departing from the spirit and scope of the invention as defined in the appended claims.

What is claimed is:

1. A cold treatment apparatus configured to cool an object comprising:
a) at least one housing;
b) at least one chiller disposed in said at least one housing;
c) at least one cold plate coupled to said at least one housing;
d) at least one controller coupled to said at least one housing, said controller configured to control a temperature of said at least one chiller; and
e) at least one handle coupled to said at least one housing.

2. The cold treatment apparatus as in claim 1, wherein said at least one housing comprises vents.

3. The cold treatment apparatus as in claim 1, further comprising a strap.

4. The cold treatment apparatus further comprising at least one battery.

5. The cold treatment apparatus wherein said at least one housing comprises at least one first side wherein said cold plate is disposed at least partially thereon, and at least one second side having vents.

6. The cold treatment apparatus as in claim 1 wherein said second side further comprises at least one central section having vents and at least one wing extending out from said at least one central section.

7. The cold treatment apparatus as in claim 6, further comprising at least one additional wing.

8. The cold treatment apparatus as in claim 7, wherein said vents are disposed on both said first side and on said second side.

9. The cold treatment apparatus as in claim 8, wherein said vents on said first side are disposed in a region adjacent to said handles.

10. The cold treatment apparatus as in claim 9, wherein said vents on said first side extend on an angle.

11. The cold treatment apparatus as in claim 1 further comprising at least one transformer which is configured to provide direct power to the device.

12. The cold treatment apparatus as in claim 1, further comprising at least one fan, wherein said at least one fan is positioned inside of the housing.

13. The cold treatment apparatus as in claim 12, wherein said at least one fan is positioned adjacent to said at least one chiller.

14. The cold treatment apparatus as in claim 13, further comprising vents, wherein said vents are configured to provide air passage into and out of the body.

15. The cold treatment apparatus as in claim 1, wherein the chiller has ribs.
